(19) United States
(12) Patent Application Publication
(10) Pub. No.: US 2011/0117195 A1
(43) Pub. Date: May 19, 2011

(54) METHOD FOR IMPROVING MYOCARDIAL INFARCTION BY INTRAMYOCARDIAL OR TRANSENDOCARDIAL INJECTION OF PEPTIDE NANOFIBERS

(75) Inventors: Ching-ho Hsieh, Tainan City (TW); Yi-dong Lin, Dali City (TW); Yu-jen Yang, Tainan (TW); Ming-long Yeh, Tainan City (TW)

(73) Assignee: National Cheng Kung University, Tainan City (TW)

(21) Appl. No.: 12/620,890

(22) Filed: Nov. 18, 2009

(51) Int. Cl. A61K 38/10; A61P 9/10; A61K 9/00; A61K 38/02; A61K 35/12

(52) U.S. Cl. 424/484; 424/93.7; 514/16.4; 514/21.2; 514/21.3; 514/21.4; 514/21.5

(57) ABSTRACT

A method for improving myocardial infarction by intramyocardial or transendocardial injection of peptide nanofibers is disclosed. The method firstly provides a pharmaceutical composition having a biologically compatible peptide hydrogel formed by a plurality of self-assembling peptide nanofibers and selectively having at least one type of autologous stem cells mixed with the self-assembling peptide nanofibers, and then the pharmaceutical composition is administered to an entire infarcted area of myocardium tissue with myocardial infarction by intramyocardial or transendocardial injection. Thus, adverse cardiac remodeling and dysfunction after acute infraction can be attenuated, while the therapeutic myocardial angiogenesis, the myocardial capillary density and potential myogenesis can be enhanced.

### FIELD OF THE INVENTION

The present invention relates to a method for improving myocardial infarction by intramyocardial injection, and more particularly to a method for improving cardiac performance after myocardial infarction by intramyocardial or transendocardial injection of peptide nanofibers or peptide nanofibers with autologous stem cells.

### BACKGROUND OF THE INVENTION

Certain peptides are capable of self assembly when incubated in the presence of a low concentration of monovalent metal cation (U.S. Pat. Nos. 5,670,483; 6,548,630). Self-assembly of the peptides results in the formation of a gel-like structure that is non-toxic, non-immunogenic and relatively stable to proteases. After peptides form hydrogels, the hydrogels are stable in serum, aqueous solutions and cell culture medium. The hydrogels are capable of supporting the growth of cells, and are slowly digested when implanted in an animal's body. Thus, the hydrogels are suitable to be used as carriers for the delivery of therapeutic agents, such as platelet-derived growth factor (PDGF).

U.S. Pat. No. 7,429,567, entitled “Sustained delivery of PDGF using self-assembling peptide nanofibers” discloses a therapeutic composition in which human PDGF is bound directly to peptides that self assemble into a biologically compatible hydrogel. When the composition of the hydrogel and the PDGF is implanted in a patient's body, the composition provides a slow, sustained release of PDGF. The composition is suitably used to treat patients who have undergone a myocardial infarction (MI), wherein MI occurs when the blood supplied to a part of the heart is interrupted and may lead to cardiomyocyte necrosis and apoptosis, while the myocardium will undergo deleterious remodeling, ultimately resulting in ventricular dilatation and pump dysfunction. When the composition is applied to treat an affected part selected from myocardial infarction, wound, damaged ligament, tendon or cartilage, or damaged nerve tissue, the method of using the composition comprises a step of administering the composition to the affected part, wherein the composition comprises a biologically compatible peptide hydrogel formed by self-assembling peptides and PDGF bound to a portion of the self-assembling peptides of the biologically compatible peptide hydrogel.

In a case that a rat model of myocardial injury is used, the nanofibers with bound PDGF can be locally injected into the border zone of an affected part of injured myocardium of a heart and are retained at the affected part for at least 14 days after coronary artery ligation. As a result, cardiomyocyte death is decreased and myocardial systolic function is maintained. Thus, the nanofibers with bound PDGF provide an effective method for preventing heart failure after myocardial infarction. However, PDGF only can promote the survival of few live cardiomyocytes remaining in the border zone of the affected part, but cannot promote the growth of non-viable (death) cardiomyocytes in the central zone of the affected part. As a result, the total nanofibers with bound PDGF were only injected into the border zone (i.e. peripheral edge) of the affected part through three directions (equal amount for each injection) immediately after coronary artery ligation without being injected into the central zone of the affected part of heart or other zone except for the border zone. If the myocardium in the central zone of the affected part is not treated, the cardiac performance (such as myocardial systolic and diastolic functions) of the affected part can not be efficiently improved.

As a result, it is important to think how to develop a method for improving myocardial infarction by intramyocardial or transendocardial injection of a suitable therapeutic composition, in order to solve the problems existing in the conventional therapeutic composition, as described above.

### SUMMARY OF THE INVENTION

An object of the present invention is to provide a method for improving myocardial infarction by intramyocardial or transendocardial injection of peptide nanofibers, wherein local intramyocardial or transendocardial injections of peptide nanofibers in the entire infarcted area of the infarcted myocardium of a heart can support the structure of the infarcted area, so as to attenuate adverse cardiac remodeling and dysfunction after acute infraction, while the intramyocardial or transendocardial injections of peptide nanofibers also can improve post-infarction diastolic functions and the cardiac performance.

Another object of the present invention is to provide a method for improving myocardial infarction by intramyocardial or transendocardial injection of peptide nanofibers with autologous stem cells, wherein the peptide nanofibers are mixed with autologous stem cells (such as bone marrow mononuclear cells), and the mixture are applied to intramyocardial or transendocardial injection in the entire infarcted area of the infarcted myocardium of the heart, so that the autologous stem cells retained within the infarcted area can be increased and the retention time of the autologous stem cells for cell therapy can be elongated. Thus, not only the pathological ventricular remodeling and the diastolic dysfunction can be efficiently prevented, but also the myocardial viability and the systolic functions can be substantially improved, while the therapeutic myocardial angiogenesis, the myocardial capillary density and potential myogenesis can be enhanced.

Further another object of the present invention is to provide a method for improving myocardial infarction by intramyocardial or transendocardial injection of peptide nanofibers, wherein the biologically compatible peptide nanofibers in the infarcted area can be further used to fasten and retain autologous peripheral blood stem cells (PBSCs) carried by blood flowing through the infarcted area of myocardium tissue or in situ endothelial stem cells of an injured heart of a patient or an animal model for therapeutic angiogenesis in the heart after the pharmaceutical composition (with autologous stem cells) is administered, so as to be also advantageous to prevent heart failure after myocardial infarction and increase the myocardial angiogenesis, the myocardial capillary density and potential myogenesis.

To achieve the above object, a preferred embodiment of the present invention provides a method for improving myocardial infarction by intramyocardial or transendocardial injection of peptide nanofibers comprising steps of: providing a pharmaceutical composition comprising a biologically compatible peptide hydrogel formed by a plurality of self-assembling peptide nanofibers with 8-200 amino acids in length, wherein the self-assembling peptide nanofibers having alternating hydrophobic and hydrophilic amino acids are complementary and structurally compatible to one another; and administering the pharmaceutical composition to an entire infarcted area of myocardium tissue with myocardial infarction by intramyocardial or transendocardial injection.

In addition, another preferred embodiment of the present invention provides a method for improving myocardial infarction by intramyocardial or transendocardial injection of peptide nanofibers with autologous stem cells comprising steps of: providing a pharmaceutical composition comprising a biologically compatible peptide hydrogel formed by a plurality of self-assembling peptide nanofibers having alternating hydrophobic and hydrophilic amino acids which are complementary and structurally compatible to one another, and at least one type of autologous stem cells mixed with the self-assembling peptide nanofibers; and administering the pharmaceutical composition to an entire infarcted area of myocardium tissue with myocardial infarction by intramyocardial or transendocardial injection.

In one aspect of the present invention, the biologically compatible peptide nanofibers are prepared by: dissolving powders of the self-assembling peptide nanofibers in a buffer solution; and mixing the powders of the self-assembling peptide nanofibers with the buffer solution by sonication, so as to obtain a hydrogel solution of the biologically compatible peptide nanofibers, wherein the percentage of the self-assembling peptide nanofibers in the solution is 0.1-10 by weight of the solution, preferably 0.5-5% by weight, and more preferably 1.0% by weight; and wherein the volume of the solution of the biologically compatible peptide hydrogel is preferably 0.1-10 ml, and more preferably 1-5 ml.

In one aspect of the present invention, the buffer solution is added with monovalent metal cation of a concentration sufficient to promote the self-assembly of the self-assembling peptide nanofibers, wherein the monovalent metal cation is selected from the group consisting of lithium (Li⁺), sodium (Na⁺) and potassium (K⁺).

In one aspect of the present invention, the pharmaceutical composition is injected to a plurality of delivery sites in the entire infarcted area of myocardium tissue, wherein the number of the delivery sites is preferably ranged between 5 and 100, and more preferably 10-50.

In one aspect of the present invention, the infarcted area of myocardium tissue is preferably a mid-left portion of myocardium tissue of a heart.

In one aspect of the present invention, the pharmaceutical composition is prepared by mixing the solution of the biologically compatible peptide nanofibers with the autologous stem cells having 10⁶-10¹⁰ cells, preferably 10⁶-10⁹ cells, and more preferably 10⁸ cells.

In one aspect of the present invention, the autologous stem cells are selected from autologous adult stem cells or autologous induced pluripotent or multipotent stem cells.

In one aspect of the present invention, the autologous adult stem cells are selected from autologous bone marrow mononuclear cells, autologous umbilical cord blood or placental stem cells, autologous peripheral blood stem cells (PBSCs), or autologous stem cells separated from fats, heart, lungs, vessels, muscles or other adult tissues.

In one aspect of the present invention, the autologous induced pluripotent or multipotent stem cells are selected from autologous somatic cells which are transformed into stem cells with the potential of differentiating into cardiomyocytes, vascular smooth muscle cells, endothelial cells or pacemaker cells for cardiac therapy using viral or non-viral transfection methods or pharmacological inducers.

In one aspect of the present invention, the biologically compatible peptide nanofibers in the infarcted area are further used to fasten and retain autologous peripheral blood stem cells (PBSCs) carried by blood flowing through the infarcted area of myocardium tissue or in situ endothelial or stem cells after the pharmaceutical composition (comprising the autologous stem cells) is administered.

In one aspect of the present invention, the self-assembling peptide nanofibers are preferably 12-32 amino acids in length, and more preferably 16 amino acids in length.

In one aspect of the present invention, the self-assembling peptide nanofibers are homogeneous.

In one aspect of the present invention, the self-assembling peptide nanofibers are selected from the group consisting of:

| | |
|---|---|
| AKAKAEAEAKAKAEAE, | (SEQ ID NO 1) |
| AKAEAKAEAKAEAKAE, | (SEQ ID NO 2) |
| EAKAEAKAEAKAEAKA, | (SEQ ID NO 3) |
| KAEAKAEAKAEAKAEA, | (SEQ ID NO 4) |
| AEAKAEAKAEAKAEAK, | (SEQ ID NO 5) |
| ADADARARADADARAR, | (SEQ ID NO 6) |
| ARADARADARADARAD, | (SEQ ID NO 7) |
| DARADARADARADARA, | (SEQ ID NO 8) |
| RADARADARADARADA, | (SEQ ID NO 9) |
| ADARADARADARADAR, | (SEQ ID NO 10) |
| ARADAKAE ARADAKAE, | (SEQ ID NO 11) |
| AKAEARADAKAKARAD, | (SEQ ID NO 12) |
| ARAKADAEARAKADAE, | (SEQ ID NO 13) |
| AKARAEADAKARAEAD, | (SEQ ID NO 14) |
| AQAQAQAQAQAQAQAQ, | (SEQ ID NO 15) |
| VQVQVQVQVQVQVQVQ, | (SEQ ID NO 16) |
| YQYQYQYQYQYQYQYQ, | (SEQ ID NO 17) |
| HQHQHQHQHQHQHQHQ, | (SEQ ID NO 18) |
| ANANANANANANANAN, | (SEQ ID NO 19) |
| VNVNVNVNVNVNVNVN, | (SEQ ID NO 20) |
| YNYNYNYNYNYNYNYN, | (SEQ ID NO 21) |
| HNHNHNHNHNHNHNHN, | (SEQ ID NO 22) |
| ANAQANAQANAQANAQ, | (SEQ ID NO 23) |
| AQANAQANAQANAQAN, | (SEQ ID NO 24) |
| VNVQVNVQVNVQVNVQ, | (SEQ ID NO 25) |
| VQVNVQVNVQVNVQVN, | (SEQ ID NO 26) |
| YNYQYNYQYNYQYNYQ, | (SEQ ID NO 27) |
| YQYNYQYNYQYNYQYN, | (SEQ ID NO 28) |
| HNHQHNHQHNHQHNHQ, | (SEQ ID NO 29) |
| HQHNHQHNHQHNHQHN, | (SEQ ID NO 30) |
| AKAQADAKAQADAKAQAD, | (SEQ ID NO 31) |
| VKVQVDVKVQVDVKVQVD, | (SEQ ID NO 32) |
| YKYQYDYKYQYDYKYQYD, | (SEQ ID NO 33) |
| HKHQHDHKHQHDHKHQHD, | (SEQ ID NO 34) |
| RARADADARARADADA, | (SEQ ID NO 35) |
| RADARGDARADARGDA, | (SEQ ID NO 36) |
| RAEARAEARAEARAEA, | (SEQ ID NO 37) |
| KADAKADAKADAKADA, | (SEQ ID NO 38) |
| AEAEAHAHAEAEAHAHA, | (SEQ ID NO 39) |
| FEFEFKFKFEFEFKFK, | (SEQ ID NO 40) |
| LELELKLKLELELKLK, | (SEQ ID NO 41) |
| AEAEAKAKAEAEAKAK, | (SEQ ID NO 42) |
| AEAEAEAEAKAK, | (SEQ ID NO 43) |
| KAKAKAKAEAEAEAEA, | (SEQ ID NO 44) |
| AEAEAEAEAKAKAKAK, | (SEQ ID NO 45) |
| RARARARADADADADA, | (SEQ ID NO 46) |
| ADADADADARARARAR, | (SEQ ID NO 47) |
| DADADADARARARARA, | (SEQ ID NO 48) |
| HEHEHKHKHEHEHKHK, | (SEQ ID NO 49) |
| VEVEVEVEVEVEVEVEVEVE, | (SEQ ID NO 50) |
| RFRFRFRFRFRFRFRFRFRF, | (SEQ ID NO 51) |

In one aspect of the present invention, the self-assembling peptide nanofibers are preferably RARADADARARADADA (SEQ ID NO 35), ADADARARADADARAR (SEQ ID NO 6), ARADARADARADARAD (SEQ ID NO 7), DARADARADARADARA (SEQ ID NO 8), RADARADARADARADA (SEQ ID NO 9), ADARADARADARADAR (SEQ ID NO 10), RARARARADADADADA (SEQ ID NO 46), ADADADADARARARAR (SEQ ID NO 47) or DADADADARARARARA (SEQ ID NO 48).

### DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a schematic view of intramyocardial injection of self-assembling peptide nanofibers (NFs) into infarcted myocardium according to a preferred embodiment of the present invention, wherein sexually matured Lanyu mini-pig models of experimental myocardial infarction (MI) is treated with peptide NF injections, and the peptide NFs form a gel-like (i.e. hydrogel) structure after sonication and become consolidated after intramyocardial injections, and wherein the mid-left anterior descending coronary artery is permanently ligated, followed with a total of 2 ml peptide NF injection in 40 delivery sites of the entire infarcted areas of the infarcted myocardium; and

FIG. 2 is a schematic view of intramyocardial injection of self-assembling peptide nanofibers with autologous stem cells into infarcted myocardium according to another preferred embodiment of the present invention, similar to FIG. 1, wherein sexually matured Lanyu mini-pig models of experimental MI is treated with peptide NF injections with autologous bone marrow mononuclear cells, and the peptide NFs form a hydrogel structure after sonication and become consolidated after intramyocardial injections, and wherein the mid-left anterior descending coronary artery is permanently ligated, followed with a total of 2 ml peptide NF injections in 40 delivery sites of the entire infarcted areas of the infarcted myocardium.

### DETAILED DESCRIPTION OF THE PREFERRED EMBODIMENTS

The present invention is related to a method for improving myocardial infarction by intramyocardial or transendocardial injection of peptide nanofibers (or peptide nanofibers with autologous stem cells), wherein a pharmaceutical composition comprising biologically compatible peptide hydrogels formed by self-assembling peptide nanofibers can be injected into an entire infarcted area of infarcted myocardium of a heart for supporting the myocardial structure of the entire infarcted area and improving the cardiac performance. Preferably, the pharmaceutical composition can further comprise at least one type of autologous stem cells which are mixed with the self-assembling peptide nanofibers and attached thereto for increasing the therapeutic myocardial angiogenesis, the myocardial capillary density and potential myogenesis. Thus, the cardiac performance of the entire infarcted area of infarcted myocardium can be improved and enhanced.

The biologically compatible peptide hydrogels formed by the self-assembling peptide nanofibers can be singly used for supporting the myocardial structure. If necessary, the biologically compatible peptide nanofibers can be mixed with an active component. For example, in the present invention, the autologous stem cells, and particularly the autologous bone marrow cells, can be mixed with and tightly attached to a portion of some self-assembling peptide nanofibers. This allows the biologically compatible peptide nanofibers to be used as a drug delivery of immobilized autologous stem cells, so that the biologically compatible peptide nanofibers can be implanted in vivo without rapidly losing the autologous stem cells. Using a sexually matured Lanyu mini-pig model of myocardial injury, the biologically compatible peptide nanofibers bound with the autologous stem cells can be locally injected into the entire infarcted area of injured myocardium and are retained at a plurality of delivery sites in the infarcted area for at least 14 days after experimental coronary artery ligation. As a result, cardiomyocyte death can be apparently decreased and myocardial function can be maintained. Thus, the biologically compatible peptide nanofibers bound with the autologous stem cells provide a more effective method for preventing heart failure after myocardial infarction. Furthermore, the biologically compatible peptide nanofibers in the infarcted area can be selectively used to further fasten and retain autologous peripheral blood stem cells (PBSCs) carried by blood flowing through the infarcted area of myocardium tissue or in situ endothelial or stem cells of an injured heart of a patient or an animal model for therapeutic angiogenesis in the heart after the pharmaceutical composition is administered, so as to be also advantageous to prevent heart failure after myocardial infarction and increase the myocardial angiogenesis, the myocardial capillary density and potential myogenesis.

In one preferable embodiment of the present invention, a biologically compatible peptide hydrogel constructed by self-assembling peptide nanofibers is applied to intramyocardial or transendocardial injection, wherein the term “biologically compatible” indicates that the hydrogel is non-toxic and can be safely implanted in a patient, while the term “hydrogel” in the present invention refers to a three dimensional solid gel-like structure. The self-assembling peptide nanofibers should be 8-200 amino acids in length and have alternating hydrophobic and hydrophilic amino acids. In addition, the peptide nanofibers are complementary for forming ionic or hydrogen bonds with one another, and are also structurally compatible, wherein the peptide chains bound to one another can maintain a distance less than about 3 angstroms throughout their length from one another. In general, 0.1-10%, more preferably 0.5-5%, and still more preferably about 1% by weight of the peptide nanofibers that assemble into the hydrogel should be bound directly to at least one type of autologous stem cells. The term “bound directly” as used herein means that there is no linker molecule or other molecules dispersed between the autologous stem cells and the peptide nanofibers. The term also requires that there be some type of physical interaction between the autologous stem cells and the peptide nanofibers (e.g. an ionic bond, hydrophobic interaction and etc.), so as to prevent the autologous stem cells from separating from the peptide nanofibers of the hydrogel in aqueous medium. Ionic bonds would form between acidic and basic amino acid side chains. The hydrophilic basic amino acids include Lys (K), Arg (R) and His (H). The hydrophilic acidic amino acids are Glu (E) and Asp (D). Ionic bonds would form between an acidic residue on one peptide and a basic residue on another. Amino acids that form hydrogen bonds are Asn (N) and Gln (Q). Hydrophobic amino acids that may be incorporated into peptide nanofibers include Ala (A), Val (V), Ile (I), Met (M), Phe (F), Tyr (Y), Trp (W), Ser (S), Thr (T), and Gly (G).

The present invention is also related to administering a pharmaceutical composition to an infarcted area of myocardium tissue of an injured heart of a patient or an animal model with myocardial infarction. It should be noted that the term “infarcted area” in the present invention refers to an affected part of myocardium tissue where myocardial infarction occurs, wherein most of cardiomyocytes in the central zone and the border zone of the affected part are almost seriously injured or died, while a few of cardiomyocytes in the border zone of the affected part are slightly injured and still viable. The term “entire infarcted area” in the present invention refers to the infarcted area considerably comprises a plurality of delivery sites which can be injected with peptide nanofibers or peptide nanofibers with autologous stem cells, wherein the number of the delivery sites is preferably ranged between 5 and 100, and more preferably 10-50, but can be suitably varied according to the size of the infarcted area. The infarcted area of myocardium tissue is preferably a mid-left portion of myocardium tissue of an injured heart with myocardial infarction, but also can be any infarcted area of the heart without limitation thereto.

The self-assembling peptide nanofibers have been described in U.S. Pat. Nos. 5,670,483 and 6,548,630, both of which are hereby incorporated by reference. Essentially the same procedures described therein for making and using the peptide nanofibers apply to the present invention. However, it has been found that the autologous stem cells can be non-covalently bound to the hydrogel formed by the self-assembling peptide nanofibers by simply combining the peptide nanofibers and the autologous stem cells in aqueous medium (e.g., water, saline or a buffer containing the components needed for the self assembly of peptide nanofibers). It appears that, 0.5-5% of the peptide nanofibers within hydrogels can be bound to the autologous stem cells but an upper limit is not limited thereto. In the preferred embodiment, the self-assembling peptide nanofibers used in hydrogels are between 8 and 200 amino acids in length, preferably between 12 and 32 amino acids in length, and more preferably 16 amino acids in length. Peptide nanofibers longer than about 200 amino acids in length tend to lower the solubility thereof and thus should be avoided. In addition, some of the self-assembling peptide nanofibers attached with the autologous stem cells are homogeneous. The term “homogeneous” as used in the present invention indicates that all of the peptide nanofibers forming the biologically compatible hydrogel are substantially identical. The term “heterogeneous” refers to non-identical peptide nanofibers that are used to form hydrogels. Specific peptide nanofibers that may be used to construct the hydrogels described above are selected from the group consisting of:

| | |
|---|---|
| AKAKAEAEAKAKAEAE, | (SEQ ID NO 1) |
| AKAEAKAEAKAEAKAE, | (SEQ ID NO 2) |
| EAKAEAKAEAKAEAKA, | (SEQ ID NO 3) |
| KAEAKAEAKAEAKAEA, | (SEQ ID NO 4) |
| AEAKAEAKAEAKAEAK, | (SEQ ID NO 5) |
| ADADARARADADARAR, | (SEQ ID NO 6) |
| ARADARADARADARAD, | (SEQ ID NO 7) |
| DARADARADARADARA, | (SEQ ID NO 8) |
| RADARADARADARADA, | (SEQ ID NO 9) |
| ADARADARADARADAR, | (SEQ ID NO 10) |
| ARADAKAEARADAKAE, | (SEQ ID NO 11) |
| AKAEARADAKAKARAD, | (SEQ ID NO 12) |
| ARAKADAEARAKADAE, | (SEQ ID NO 13) |
| AKARAEADAKARAEAD, | (SEQ ID NO 14) |
| AQAQAQAQAQAQAQAQ, | (SEQ ID NO 15) |
| VQVQVQVQVQVQVQVQ, | (SEQ ID NO 16) |
| YQYQYQYQYQYQYQYQ, | (SEQ ID NO 17) |
| HQHQHQHQHQHQHQHQ, | (SEQ ID NO 18) |
| ANANANANANANANAN, | (SEQ ID NO 19) |
| VNVNVNVNVNVNVNVN, | (SEQ ID NO 20) |
| YNYNYNYNYNYNYNYN, | (SEQ ID NO 21) |
| HNHNHNHNHNHNHNHN, | (SEQ ID NO 22) |
| ANAQANAQANAQANAQ, | (SEQ ID NO 23) |
| AQANAQANAQANAQAN, | (SEQ ID NO 24) |
| VNVQVNVQVNVQVNVQ, | (SEQ ID NO 25) |
| VQVNVQVNVQVNVQVN, | (SEQ ID NO 26) |
| YNYQYNYQYNYQYNYQ, | (SEQ ID NO 27) |
| YQYNYQYNYQYNYQYN, | (SEQ ID NO 28) |
| HNHQHNHQHNHQHNHQ, | (SEQ ID NO 29) |
| HQHNHQHNHQHNHQHN, | (SEQ ID NO 30) |
| AKAQADAKAQADAKAQAD, | (SEQ ID NO 31) |
| VKVQVDVKVQVDVKVQVD, | (SEQ ID NO 32) |
| YKYQYDYKYQYDYKYQYD, | (SEQ ID NO 33) |
| HKHQHDHKHQHDHKHQHD, | (SEQ ID NO 34) |
| RARADADARARADADA, | (SEQ ID NO 35) |
| RADARGDARADARGDA, | (SEQ ID NO 36) |
| RAEARAEARAEARAEA, | (SEQ ID NO 37) |
| KADAKADAKADAKADA, | (SEQ ID NO 38) |
| AEAEAHAHAEAEAHAHA, | (SEQ ID NO 39) |
| FEFEFKFKFEFEFKFK, | (SEQ ID NO 40) |
| LELELKLKLELELKLK, | (SEQ ID NO 41) |
| AEAEAKAKAEAEAKAK, | (SEQ ID NO 42) |
| AEAEAEAEAKAK, | (SEQ ID NO 43) |
| KAKAKAKAEAEAEAEA, | (SEQ ID NO 44) |
| AEAEAEAEAKAKAKAK, | (SEQ ID NO 45) |
| RARARARADADADADA, | (SEQ ID NO 46) |
| ADADADADARARARAR, | (SEQ ID NO 47) |
| DADADADARARARARA, | (SEQ ID NO 48) |
| HEHEHKHKHEHEHKHK, | (SEQ ID NO 49) |
| VEVEVEVEVEVEVEVEVEVE, | (SEQ ID NO 50) |
| RFRFRFRFRFRFRFRFRFRF, | (SEQ ID NO 51) |

It should be noted that each of the peptide nanofibers listed above includes a repeating sequence and that additional repeats can be included to extend the length of the peptide nanofibers without affecting the property of self-assembly. For example, the peptide AKAKAEAEAKAKAEAE (SEQ ID NO:1) has the repeating sequence AKAKAEAE and can be expressed as (AKAKAEAE)ₙ, wherein n=2. Longer peptide nanofibers capable of self assembly can be made by increasing n, but the total number of amino acids in the final peptide cannot exceed 200.

Other peptide nanofibers expressed in this manner and useful in the invention are: (AKAKAEAE)ₙ, wherein n=1-25; (AKAE)ₙ, wherein n=2-50; (EAKA)ₙ, wherein n=2-50; (KAEA)ₙ, wherein n=2-50; (AEAK)ₙ, wherein n=2-50; (ADADARAR)ₙ, wherein n=1-25; (ARAD)ₙ, wherein n=2-50; (DARA)ₙ, wherein n=2-50; (RADA)ₙ, wherein n=2-50; (ADAR)ₙ, wherein n=2-50; (ARADAKAE)ₙ, wherein n=1-25; (AKAEARAD)ₙ, wherein n=1-25; (ARAKADAE)ₙ, wherein n=1-25; (AKARAEAD)ₙ, wherein n=1-25; (AQ)ₙ, wherein n=4-100; (VQ)ₙ, wherein n=4-100; (YQ)ₙ, wherein n=4-100; (HQ)ₙ, wherein n=4-100; (AN)ₙ, wherein n=4-100; (VN)ₙ, wherein n=4-100; (YN)ₙ, wherein n=4-100; (HN)ₙ, wherein n=4-100; (ANAQ)ₙ, wherein n=2-50; (AQAN)ₙ, wherein n=2-50; (VNVQ)ₙ, wherein n=2-50; (VQVN)ₙ, wherein n=2-50; (YNYQ)ₙ, wherein n=2-50; (YQYN)ₙ, wherein n=2-50; (HNHQ)ₙ, wherein n=2-50; (HQHN)ₙ, wherein n=2-50; (AKAQAD)ₙ, wherein n=2-33; (VKVQVD)ₙ, wherein n=2-33; (YKYQYD)ₙ, wherein n=2-33; (HKHQHD)ₙ, wherein n=2-33; (RARADADA)ₙ, wherein n=1-25; (RADARGDA)ₙ, wherein n=1-25; (RAEA)ₙ, wherein n=2-50; (KADA)ₙ, wherein n=2-50; (AEAEAHAH)ₙ, wherein n=1-25; (FEFEFKFK)ₙ, wherein n=1-25; (LELELKLK)ₙ, wherein n=1-25; (AEAEAKAK)ₙ, wherein n=1-25; (AEAEAEAEAKAK)ₙ, wherein n=1-16; (KAKAKAKAEAEAEAEA)ₙ, wherein n=1-12; (AEAEAEAEAKAKAKAK)ₙ, wherein n=1-12; (RARARARADADADADA)ₙ, wherein n=1-12; (ADADADADARARARAR), wherein n=1-12; (DADADADARARARARA)ₙ, wherein n=1-12; (HEHEHKHK)ₙ, wherein n=1-25; (VE)ₙ, wherein n=4-100; and (RF)ₙ, wherein n=4-100.

Preferred peptide nanofibers are those having the following repeating structures: (RARADADA)ₙ, wherein n=1-10, preferably n=2-4, and more preferably n=2, i.e. RARADADARARADADA, (SEQ ID NO 35); (ADADARAR)ₙ, wherein n=1-25, e.g. ADADARARADADARAR, (SEQ ID NO 6); (ARAD)ₙ, wherein n=2-50, e.g. ARADARADARADARAD, (SEQ ID NO 7); (DARA)ₙ, wherein n=2-50, e.g. DARADARADARADARA, (SEQ ID NO 8); (RADA)ₙ, wherein n=2-50, e.g. RADARADARADARADA, (SEQ ID NO 9); (ADAR)ₙ, wherein n=2-50, e.g. ADARADARADARADAR, (SEQ ID NO 10); (RARARARADADADADA)ₙ, wherein n=1-12, e.g. RARARARADADADADA, (SEQ ID NO 46); (ADADADADARARARAR), wherein n=1-12, e.g. ADADADADARARARAR, (SEQ ID NO 47); (DADADADARARARARA)ₙ, wherein n=1-12, e.g. DADADADARARARARA, (SEQ ID NO 48).

In the present invention, the self-assembling peptide nanofibers must also be structurally compatible for maintaining an essentially constant distance between one another when binding one another to self-assemble the hydrogel. Inter-peptide distance can be calculated for each ionized or hydrogen bonding pair by taking the sum of the number of unbranched atoms on the side-chains of each amino acid in the pair. For example, lysine has five unbranched atoms on its side chains, and glutamic acid has four unbranched atoms on its side chains. An interaction between these two residues on different peptide nanofibers would result in an interpeptide distance of nine atoms. In a peptide containing only repeating units of EAK, all of the ion pairs would involve lysine (K) and glutamate (E), so that a constant interpeptide distance would be maintained. Thus, these peptide nanofibers would be structurally complementary to one another. Peptide nanofibers in which the variation in interpeptide distance is more than one atom (about 3-4 angstroms) will not properly form a hydrogel structure. For example, if two bound peptide nanofibers have ion pairs with a nine-atom spacing and other ion pairs with a seven-atom spacing, the requirement of structural complementarity can not have been met, wherein other discussion of complementarity and structural compatibility can be found in U.S. Pat. Nos. 5,670,483 and 6,548,630. The definitions used therein and examples provided can be applied equally to the present invention.

It should also be noted that the hydrogels may be formed from either a homogeneous mixture of peptide nanofibers or a heterogeneous mixture of peptide nanofibers. The term “homogeneous” in the present invention means peptide nanofibers that are identical to one another. The term “heterogeneous” indicates peptide nanofibers that bind to one another but which are structurally different from one another. Regardless of whether homogenous or heterogeneous peptide nanofibers are used, the requirements with respect to the arrangement of amino acids, length, complementarity, and structural compatibility must apply. In addition, it should be noted that the carboxyl and amino groups of the terminal residues of peptide nanofibers can either be protected or not protected using standard groups.

The self-assembling peptide nanofibers of the present invention can be formed by solid-phase peptide synthesis using standard N-tert-butoxycarbonyl (t-Boc) chemistry and cycles using n-methylpyrolidone chemistry. Once peptide nanofibers have been synthesized, they can be purified using procedures such as high pressure liquid chromatography on reverse-phase columns. Purity may also be assessed by HPLC (high-performance liquid chromatography) and the presence of a correct composition can be determined by amino acid analysis.

The self-assembling peptide nanofibers described herein will not form hydrogels in water, but will self-assemble in an aqueous solution (such as a buffer solution) containing monovalent metal cation of a low concentration. The order of effectiveness of these cations is Li⁺>Na⁺>K⁺>Cs⁺ (U.S. Pat. No. 6,548,630). A concentration of monovalent metal cation of 5 mM is sufficient for peptide nanofibers to self-assemble, and the concentration as high as 5 M still can be effective. The anion associated with the monovalent metal cation is not critical to the present invention and can be hydroxide, acetate, chloride, sulfate, phosphate, etc. If the autologous stem cells are used, the autologous stem cells will bind to the peptide nanofibers at low salt concentration and will remain bound at concentrations sufficient to induce self assembly.

The initial concentration of peptide nanofibers will influence the final size and thickness of the hydrogel formed therefrom. In general, when the peptide concentration is increase, the extent of hydrogel formation can be expanded. The hydrogel can be formed in peptide concentration as low as 0.1-10% by weight of the solution, preferably 0.5-5% by weight, and more preferably 1.0% by weight. However, the hydrogel is preferably formed at a higher initial peptide concentration, such as 1.0% by weight (10 mg/ml). Moreover, it is generally better to form the hydrogel by adding the peptide nanofibers to a salt solution or buffer solution rather than adding salt or buffer to a peptide solution.

The formation of the hydrogel is relatively unaffected by pH or by temperature. Nevertheless, pH should be maintained below 12 and temperatures should generally be in the range of 4-90° C. Monovalent metal cation at a concentration of 5 mM is sufficient for peptide nanofibers to assemble into the hydrogel. Hydrogel formation may be observed by simple visual inspection and this can be aided, if desired, with stains such as Congo Red. The integrity of the hydrogel can also be observed microscopically, with or without stain.

Stem cells (such as mesenchymal stem cells) can be found in the bone marrow or in other autologous tissues of adult humans. Stem cells have the potential to differentiate and develop into mature cells of fat, cartilage, bone, tendon, nerve, muscle (such as cardiomyocytes or smooth muscle cells, SMCs) or endothelial cells (EC). In the present invention, stem cells can be isolated from human autologous bone marrow, fat, umbilical cord blood and etc., and transferred into the entire infarcted area of an infarcted myocardium of a heart via local intramyocardial or transendocardial injections, so as to grow and reproduce and even still maintain the stem cell capabilities. Autologous bone marrow mononuclear cells or autologous umbilical cord blood stem cells isolated as described above is advantageous to increase the therapeutic myocardial angiogenesis, the myocardial capillary density and potential myogenesis in the infarcted myocardium.

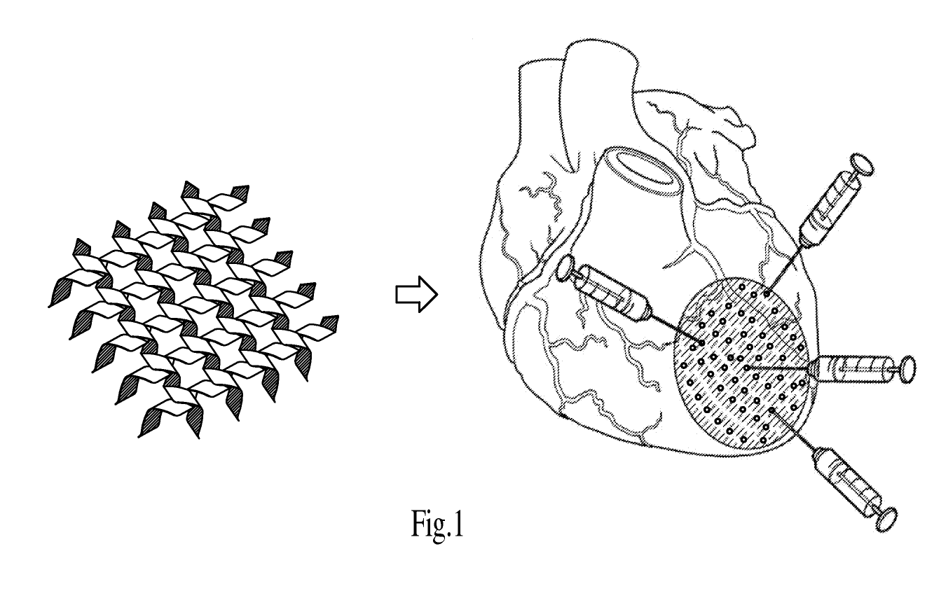

Referring now to FIG. 1, a method for improving myocardial infarction by intramyocardial injection of peptide nanofibers (or peptide nanofibers with autologous stem cells) according to a preferred embodiment of the present invention is illustrated, wherein the method comprises steps of: providing a pharmaceutical composition comprising a biologically compatible peptide hydrogel formed by a plurality of self-assembling peptide nanofibers with 8-200 amino acids in length, wherein the self-assembling peptide nanofibers having alternating hydrophobic and hydrophilic amino acids are complementary and structurally compatible to one another; and administering the pharmaceutical composition to an infarcted area of myocardium tissue with myocardial infarction by intramyocardial or transendocardial injection. As shown, an intramyocardial injection of self-assembling peptide nanofibers (NFs) into infarcted myocardium is carried out according to the preferred embodiment of the present invention, wherein sexually matured Lanyu mini-pigs model of experimental myocardial infarction (MI) is treated with peptide NFs injection, and the peptide NFs forms a gel-like (i.e. hydrogel) structure after sonication and becomes consolidated after intramyocardial injection, and wherein the mid-left anterior descending coronary artery is permanently ligated, followed with 50 ul peptide NFs injection in 40 delivery sites of the entire infarcted areas of the infarcted myocardium (total dose: 2 ml). The method of the preferred embodiment will be described more detailed hereinafter.

Materials and Methods

Sexually matured Lanyu mini-pigs (about 5 months old, body weight: 21.8±3.1 kg), were divided into 3 groups: the first group is sham operation, suturing was performed without ligation (n=4); the second group is mid-left coronary artery ligation (for simulating myocardial infarction, MI) immediately followed with injection of normal saline solution (MI+NS, n=4); and the third group is mid-left coronary artery ligation immediately followed with injection of 1% peptide nanofibers solution (MI+NFs, n=5) in the entire infarcted area with a total of 2 ml NFs divided by 40 delivery sites (50 ul for each site), wherein the sequence of peptide NFs is AcN-RARADADARARADADA-NH₂. Cardiac functions were assessed by echocardiography immediately after MI and together with cardiac catheterization 4 weeks later.

In the preferred embodiment of the present invention, the peptide nanofibers solution contains peptide nanofibers which can form a biologically compatible peptide hydrogel, wherein the biologically compatible peptide hydrogel is prepared by: dissolving powders of the self-assembling peptide nanofibers in a buffer solution, such as pH 7.4 phosphate buffered saline (PBS) solution; and mixing the powders of the self-assembling peptide nanofibers with the buffer solution by sonication (100 W, 10 mins), so as to obtain a hydrogel solution of the biologically compatible peptide hydrogel (i.e. the peptide nanofibers solution), wherein the percentage of the self-assembling peptide nanofibers in the solution can be 0.1-10% by weight of the solution, preferably 0.5-5% by weight, and more preferably 1.0% by weight; and wherein the volume of the solution of the biologically compatible peptide hydrogel is preferably 0.1-10 ml, and more preferably 2 ml. In the embodiment, the percentage of the self-assembling peptide nanofibers in the solution is 1.0% by weight, and the volume of the solution of the biologically compatible peptide hydrogel is 2 ml. Meanwhile, the buffer solution (PBS) is added with monovalent metal cation compound of a concentration sufficient to promote the self-assembly of the self-assembling peptide nanofibers, wherein the monovalent metal cation is selected from the group consisting of: lithium (Li⁺), sodium (Na⁺) and potassium (K⁺). In the embodiment, the monovalent metal cation compound is sodium hydroxide (NaOH).

Besides, the pharmaceutical composition of the second and third groups is injected to a plurality of delivery sites in the entire infarcted area of myocardium tissue, respectively, wherein the number of the delivery sites is preferably ranged between 10 and 100, and more preferably 40, without limitation thereto. The plurality of delivery sites in the entire infarcted area is advantageous to increase the distribution density to support materials (e.g. peptide nanofibers). In addition, the infarcted area of myocardium tissue is preferably a mid-left portion of myocardium tissue of an injured heart with myocardial infarction, but also can be any infarcted area of the heart. The self-assembling peptide nanofibers (AcN-RARADADARARADADA-NH₂) are 16 amino acids in length, but also can be 8-200 amino acids in length, and preferably 12-32 amino acids in length. However, only if the self-assembling peptide nanofibers can construct the biologically compatible peptide hydrogel, the self-assembling peptide nanofibers can be selected from at least one type of homogeneous or heterogeneous peptide nanofibers, such as peptide nanofibers of (SEQ ID NO 1) to (SEQ ID NO 51), preferably RARADADARARADADA (SEQ ID NO 35), ADADARARADADARAR (SEQ ID NO 6), ARADARADARADARAD (SEQ ID NO 7), DARADARADARADARA (SEQ ID NO 8), RADARADARADARADA (SEQ ID NO 9), ADARADARADARADAR (SEQ ID NO 10), RARARARADADADADA (SEQ ID NO 46), ADADADADARARARAR (SEQ ID NO 47) or DADADADARARARARA (SEQ ID NO 48), and more preferably RARADADARARADADA (SEQ ID NO 35).

Results

At one month after MI, there was only modest improvement in systolic function such as the ejection fraction (E.F.) and +dP/dt Max (change in pressure/change in time) of the (MI+NFs) group compared to the (MI+NS) group (no significant difference). However, the diastolic function and ventricular remodeling of the (MI+NFs) group are significantly improved, evidenced by the changes of hemodynamic parameters, including the end-diastolic and end-systolic volumes, peak pressure −dP/dt Max, and maximal chamber elasticity (Emax), as shown in Table 1. The thickness of inter-ventricular septum on diastolic (IVS-D) and systolic (IVS-S) phase of the (MI+NFs) group is also increased (diastolic: 0.49±0.08 mm and systolic: 0.55±0.07 mm for MI+NS group; and diastolic: 0.60±0.12 mm and systolic: 0.70±0.13 mm for MI+NFs group; P<0.01). The myocardial capillary density (M.C.D., number/mm²) of the (MI+NFs) group also can be significantly increased.

TABLE 1
Indexes of cardiac function at 28 days after infarction in mini-pig models

| Hemodynamic Parameter | +dP/dt Max | −dP/dt Max | Emax |
|---|---|---|---|
| Sham (n = 4) | 1699 ± 248 | −2130 ± 382 | 4.1 ± 3.4 |
| MI + NS (n = 4) | 1377 ± 347 | −1361 ± 368 | 1.48 ± 0.63 |
| MI + NFs (n = 5) | 1458 ± 154 | −1850 ± 94* | 2.80 ± 1.43 |

| Echocardiac Index | E.F. | IVS-D | IVS-S |
|---|---|---|---|
| Sham (n = 4) | 62 ± 6 | 0.61 ± 0.08 | 0.79 ± 0.02 |
| MI + NS (n = 4) | 46 ± 6 | 0.49 ± 0.08 | 0.55 ± 0.07 |
| MI + NFs (n = 5) | 47 ± 8 | 0.60 ± 0.12** | 0.70 ± 0.13** |

| Immunohistological Index | M.C.D. |
|---|---|
| Sham (n = 4) | 1385 ± 450 |
| MI + NS (n = 4) | 229 ± 119 |
| MI + NFs (n = 5) | 286 ± 179** |

*P < 0.05,
**P < 0.01;
Sham operation was performed without ligation (MI).

As described above, in the preferred embodiment of the present invention, when intramyocardial injection of peptide nanofibers into the 40 delivery sites (or delivery sites with suitable distribution density) in the entire infarcted area of injured myocardium (MI+NFs group) of an injured heart is carried out, the injections of peptide nanofibers can support the structure of the infarcted area, while the biologically compatible peptide hydrogel in the infarcted area can be used to fasten and retain autologous peripheral blood stem cells (PBSCs) carried by blood flowing through the infarcted area of myocardium tissue or in situ the endothelial or stem cells for therapeutic myocardial angiogenesis, the myocardial capillary density and potential myogenesis in the heart after the pharmaceutical composition is administered. As a result, the post-infarction diastolic functions and the cardiac performance can be improved and ventricular remodeling can be prevented in a mini-pig model of coronary ligation at the mid-left anterior descending coronary artery, while growing evidence indicated that MI+NFs group also can improve related cardiac functions and attenuate adverse cardiac remodeling and dysfunction after myocardial infarction (MI).

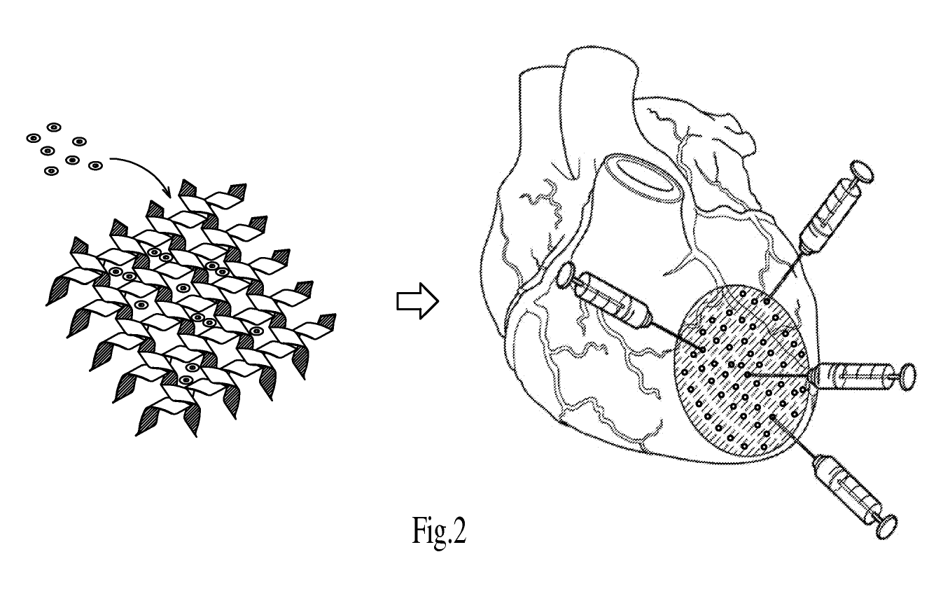

On the other hand, referring now to FIG. 2, an alternative method for improving myocardial infarction by intramyocardial injection of peptide nanofibers with autologous stem cells according to another embodiment of the present invention is illustrated and comprises steps of: providing a pharmaceutical composition comprising a biologically compatible peptide hydrogel formed by a plurality of self-assembling peptide nanofibers with 8-200 amino acids in length, wherein the self-assembling peptide nanofibers having alternating hydrophobic and hydrophilic amino acids are complementary and structurally compatible to one another, and at least one type of autologous stem cells mixed with the self-assembling peptide nanofibers; and administering the pharmaceutical composition to an infarcted area of myocardium tissue with myocardial infarction by intramyocardial injection. In the embodiment, the pharmaceutical composition further comprises at least one type of autologous stem cells mixed with the self-assembling peptide nanofibers, wherein the autologous stem cells can be selected from autologous adult stem cells or induced pluripotent/multipotent stem cells, and wherein the autologous adult stem cells are preferably selected from autologous bone marrow mononuclear cells, autologous umbilical cord blood or placental stem cells, autologous peripheral blood stem cells (PBSCs), or autologous stem cells separated from fats, heart, lungs, vessels, muscles, or other adult tissues. In addition, the autologous induced pluripotent or multipotent stem cells are selected from autologous somatic cells which are transformed into stem cells with the potential of differentiating into cardiomyocytes, vascular smooth muscle cells, endothelial cells or pacemaker cells for cardiac therapy using viral or non-viral gene transfection methods or pharmacological inducers.

In the embodiment, autologous bone marrow mononuclear cells are exemplified, and the separation method thereof comprises steps of: adding 0.5 ml of heparin into two 10 ml syringes, respectively, and then drawing out autologous bone marrow tissues from an autologous bone (such as the tibia bone) of the sexually matured Lanyu mini-pigs; placing the autologous bone marrow tissues into a 50 ml tube, and diluting by 20 ml of phosphate buffered saline (PBS) containing 5% fetal bovine serum (FBS); evenly mixing the diluted solution, and filtering through a 70 um strainer; preparing eight 10 ml tubes, and adding 5 ml of Ficoll medium into each of the tubes, respectively; slowly and averagely adding 10 ml of the diluted solution of the autologous bone marrow tissues into the Ficoll tubes without mixing the diluted solution with the Ficoll medium; centrifuging the Ficoll tubes at 2400 rpm for 15 mins; observing the location of mononuclear cell layer after centrifugation, followed by removing the supernatant above the mononuclear cell layer and collecting the mononuclear cell layer; washing the mononuclear cells three times by PBS at 1800 rpm for 2 mins; counting the cell number of the mononuclear cells and adjusting the concentration of the mononuclear cells to a suitable value after washing; and adding 2 ml of PBS+NFs (nanofibers) into the mononuclear cells having a predetermined cell number, and drawing the solution by a syringe for intramyocardial injection.

As shown in FIG. 2, an intramyocardial injection of self-assembling peptide nanofibers with the autologous bone marrow mononuclear cells (NFs+BM) into infarcted myocardium is carried out according to the embodiment, wherein sexually matured Lanyu mini-pigs model of experimental myocardial infarction (MI) is treated with peptide NFs injection containing the autologous bone marrow mononuclear cells having a predetermined cell number, and the peptide NFs forms a gel-like (i.e. hydrogel) structure after sonication and becomes consolidated after intramyocardial injection, and wherein the mid-left anterior descending coronary artery is permanently ligated, followed with 50 ul peptide NFs injection in 40 delivery sites (or delivery sites with suitable distribution density) of the entire infarcted areas of the infarcted myocardium (total dose: 2 ml). The materials and methods of the embodiment are substantially similar to that of the foregoing preferred embodiment, so that most of the detailed description will be omitted, wherein sexually matured Lanyu mini-pigs (about 5 months old, body weight: 21.8±3.1 kg) were divided into 4 groups: the first group is sham operation, suturing was performed without ligation (n=4); the second group is mid-left coronary artery ligation (for simulating myocardial infarction, MI) immediately followed with injection of normal saline solution (MI+NS, n=5); the third group is mid-left coronary artery ligation immediately followed with injection of a solution containing the autologous bone marrow mononuclear cells having 10⁸ cells (MI+BM, n=6) in the entire infarcted area with a total of 2 ml NFs divided by 40 delivery sites (50 ul for each site); and the fourth group is mid-left coronary artery ligation immediately followed with injection of 1% peptide nanofibers solution containing the autologous bone marrow mononuclear cells having 10⁸ cells (MI+NFs+BM, n=6) in the entire infarcted area with a total of 2 ml NFs divided by 40 delivery sites (50 ul for each site), wherein the sequence of peptide NFs is AcN-RARADADARARADADA-NH₂, and the cell number of the autologous stem cells is not limited to 10⁸ cells, such as the cell number may be 10⁵-10¹⁰ cells, and preferably 10⁶-10⁹ cells. Cardiac functions were assessed by echocardiography immediately after MI and together with cardiac catheterization 4 weeks later, as shown in Table 2.

TABLE 2
Indexes of cardiac function at 28 days after infarction in mini-pig models

| Hemodynamic Parameter | +dP/dt Max | −dP/dt Max | Emax |
|---|---|---|---|
| Sham (n = 5) | 1763 ± 244 | −2558 ± 453 | 4.17 ± 4.2 |
| MI + NS (n = 5) | 1217 ± 186 | −1141 ± 232 | 1.33 ± 0.62 |
| MI + BM (n = 6) | 1561 ± 357* | −1440 ± 334 | 3.24 ± 1.55 |
| MI + NFs + BM (n = 6) | 1698 ± 168* | −1886 ± 633* | 3.26 ± 0.54 |

| Echocardiac | | | |
|---|---|---|---|
| Index | E.F. | IVS-D | IVS-S |
| Sham (n = 5) | 63 ± 7 | 0.62 ± 0.07 | 0.81 ± 0.08 |
| MI + NS | 43 ± 6 | 0.48 ± 0.08 | 0.56 ± 0.06 |
| (n = 5) | | | |
| MI + BM | 50 ± 6* | 0.51 ± 0.04 | 0.66 ± 0.06* |
| (n = 6 | | | |
| MI + NFs + | 60 ± 3*** | 0.62 ± 0.05** | 0.78 ± 0.01*** |
| BM (n = 6) | | | |

| Immunohistological Index | M.C.D. |
|---|---|
| Sham (n = 5) | 1500 ± 450 |
| MI + NS (n = 5) | 230 ± 40 |
| MI + BM (n = 6 | 385 ± 203*** |
| MI + NFs + BM (n = 6) | 396 ± 189*** |

*P < 0.05,
**P < 0.01,
***P < 0.01;
Sham operation was performed without ligation (MI).

Apparently, there was further significant difference between the (MI+NS) group and the (MI+NFs+BM) group in the systolic functional parameters such as the ejection fraction and +dP/dt Max (change in pressure/change in time). In addition, the diastolic function and ventricular remodeling of the (MI+NFs+BM) group are also significantly improved, evidenced by the changes of hemodynamic parameters, including the end-diastolic and end-systolic volumes, peak pressure −dP/dt Max, and maximal chamber elasticity (Emax). The thickness of inter-ventricular septum on diastolic and systolic phase of the (MI+NFs+BM) group and the myocardial capillary density (M.C.D., number/mm²) thereof are also increased.

In the embodiment, when intramyocardial injection of peptide nanofibers with the autologous stem cells is carried out, wherein the peptide nanofibers are mixed with autologous stem cells (such as bone marrow mononuclear cells), and the mixture are applied to intramyocardial injection into the 40 delivery sites (or delivery sites with suitable distribution density) in the entire infarcted area of the infarcted myocardium of the heart, so that the autologous stem cells retained within the infarcted area can be increased and the retention time of the autologous stem cells for cell therapy can be elongated. Thus, not only the pathological ventricular remodeling and the diastolic dysfunction can be efficiently prevented, but also the myocardial viability and the systolic functions can be substantially improved, while the therapeutic myocardial angiogenesis, the myocardial capillary density and potential myogenesis in the pig model or patients can be enhanced. Furthermore, the biologically compatible peptide hydrogel in the infarcted area can be used to fasten and retain autologous peripheral blood stem cells (PBSCs) carried by blood flowing through the infarcted area of myocardium tissue or the in situ endothelial or stem cells of an injured heart of a patient or a animal model for therapeutic angiogenesis in the heart after the pharmaceutical composition comprising the autologous stem cells (such as the autologous bone marrow mononuclear cells or the autologous umbilical cord blood stem cells) is administered, so as to be also advantageous to secondarily prevent heart failure after myocardial infarction and increase the myocardial angiogenesis, the myocardial capillary density and potential myogenesis.

As described above, in comparison with the traditional sustained delivery of PDGF using self-assembling peptide nanofibers in which PDGF only can promote the growth of live cardiomyocyte tissue remaining in the border zone of the affected part and cannot promote the growth of death cardiomyocyte tissue in the central zone of the affected part, the method for improving myocardial infarction by intramyocardial or transendocardial injection of peptide nanofibers of the present invention, as shown in FIGS. 1 and 2, the intramyocardial or transendocardial injection of peptide nanofibers (with the autologous stem cells, such as bone marrow mononuclear cells) is carried out at the 40 delivery sites (or delivery sites with suitable distribution density) over the entire infarcted area of the infarcted myocardium of the heart, few live cardiomyocytes remaining in the border zone and almost death cardiomyocytes in the central zone over the entire infarcted area can be physically supported by the biologically compatible peptide hydrogel formed by the self-assembling peptide nanofibers, while the biologically compatible peptide hydrogel can be used to fasten and retain autologous stem cells, such as autologous adult stem cells, autologous induced pluripotent or multipotent stem cells or autologous peripheral blood stem cells (PBSCs) carried by blood flowing through the infarcted area or in situ endothelial or stem cells. As a result, the pathological ventricular remodeling and the diastolic dysfunction can be efficiently prevented, while the myocardial viability and the systolic functions can be substantially improved. Furthermore, the autologous stem cells retained within the infarcted area can be increased and the retention time of the autologous stem cells for cell therapy can be elongated, so that the therapeutic myocardial angiogenesis, the myocardial capillary density and potential myogenesis can be enhanced. Therefore, a potential clinical therapy for cardiac injury using intramyocardial or potentially, transendocardial injection of peptide nanofibers with autologous stem cells can be carried out.

The present invention has been described with a preferred embodiment thereof and it is understood that many changes and modifications to the described embodiment can be carried out without departing from the scope and the spirit of the invention that is intended to be limited only by the appended claims.

1. A method for improving myocardial infarction by intramyocardial or transendocardial injection of peptide nanofibers, comprising:
providing a pharmaceutical composition comprising a biologically compatible peptide hydrogel formed by a plurality of self-assembling peptide nanofibers with 8-200 amino acids in length, wherein the self-assembling peptide nanofibers having alternating hydrophobic and hydrophilic amino acids are complementary and structurally compatible to one another; and
administering the pharmaceutical composition to an entire infarcted area of myocardium tissue with myocardial infarction by intramyocardial or transendocardial injection.

2. The method for improving myocardial infarction by intramyocardial or transendocardial injection of peptide nanofibers according to claim 1, wherein the biologically compatible peptide hydrogel is prepared by:
dissolving powders of the self-assembling peptide nanofibers in a buffer solution; and
mixing the powders of the self-assembling peptide nanofibers with the buffer solution by sonication, so as to obtain a hydrogel solution of the biologically compatible peptide hydrogel.

3. The method for improving myocardial infarction by intramyocardial or transendocardial injection of peptide nanofibers according to claim 2, wherein the percentage of the self-assembling peptide nanofibers in the solution is 0.1-10% by weight of the solution, and the volume of the solution of the biologically compatible peptide hydrogel is 0.1-10 ml.

4. The method for improving myocardial infarction by intramyocardial or transendocardial injection of peptide nanofibers according to claim 2, wherein the buffer solution is added with monovalent metal cation of a concentration sufficient to promote the self-assembly of the self-assembling peptide nanofibers.

5. The method for improving myocardial infarction by intramyocardial or transendocardial injection of peptide nanofibers according to claim 4, wherein the monovalent metal cation is selected from the group consisting of lithium, sodium and potassium.

6. The method for improving myocardial infarction by intramyocardial or transendocardial injection of peptide nanofibers according to claim 1, wherein the pharmaceutical composition is injected to a plurality of delivery sites in the entire infarcted area of myocardium tissue.

7. The method for improving myocardial infarction by intramyocardial or transendocardial injection of peptide nanofibers according to claim 6, wherein the number of the delivery sites is ranged between 10 and 100.

8. The method for improving myocardial infarction by intramyocardial or transendocardial injection of peptide nanofibers according to claim 1, wherein the infarcted area of myocardium tissue is a mid-left portion of myocardium tissue of a heart.

9. The method for improving myocardial infarction by intramyocardial or transendocardial injection of peptide nanofibers according to claim 1, wherein the biologically compatible peptide hydrogel in the infarcted area fastens and retains autologous peripheral blood stem cells carried by blood flowing through the infarcted area of myocardium tissue or in situ endothelial or stem cells after the pharmaceutical composition is administered.

10. The method for improving myocardial infarction by intramyocardial or transendocardial injection of peptide nanofibers according to claim 1, wherein the pharmaceutical composition further comprises at least one type of autologous stem cells mixed with the self-assembling peptide nanofibers.

11. The method for improving myocardial infarction by intramyocardial or transendocardial injection of peptide nanofibers according to claim 10, wherein the pharmaceutical composition is prepared by mixing a solution of the biologically compatible peptide hydrogel with the autologous stem cells having 10⁵-10¹⁰ cells.

12. The method for improving myocardial infarction by intramyocardial or transendocardial injection of peptide nanofibers according to claim 10, wherein the autologous stem cells are selected from autologous adult stem cells or autologous induced pluripotent or multipotent stem cells.

13. The method for improving myocardial infarction by intramyocardial or transendocardial injection of peptide nanofibers according to claim 12, wherein the autologous adult stem cells are selected from autologous bone marrow mononuclear cells, autologous umbilical cord blood or placental stem cells, autologous peripheral blood stem cells, or autologous stem cells separated from fats, heart, lungs, vessels, muscles or other adult tissues.

14. The method for improving myocardial infarction by intramyocardial or transendocardial injection of peptide nanofibers according to claim 12, wherein the autologous induced pluripotent or multipotent stem cells are selected from autologous somatic cells which are transformed into stem cells with the potential of differentiating into cardiomyocytes, vascular smooth muscle cells, endothelial cells or pacemaker cells for cardiac therapy using viral or non-viral gene transfection methods or pharmacological inducers.

15. The method for improving myocardial infarction by intramyocardial or transendocardial injection of peptide nanofibers according to claim 10, wherein the biologically compatible peptide hydrogel in the infarcted area fastens and retains autologous peripheral blood stem cells carried by blood flowing through the infarcted area of myocardium tissue or in situ endothelial or stem cells after the pharmaceutical composition comprising the autologous stem cells is administered.

16. The method for improving myocardial infarction by intramyocardial or transendocardial injection of peptide nanofibers according to claim 1, wherein the self-assembling peptide nanofibers are 12-32 amino acids in length.

17. The method for improving myocardial infarction by intramyocardial or transendocardial injection of peptide nanofibers according to claim 1, wherein the self-assembling peptide nanofibers are homogeneous.

18. The method for improving myocardial infarction by intramyocardial or transendocardial injection of peptide nanofibers according to claim 1, wherein the self-assembling peptide nanofibers are selected from the group consisting of:

| | |
|---|---|
| AKAKAEAEAKAKAEAE,; | (SEQ ID NO 1) |
| AKAEAKAEAKAEAKAE,; | (SEQ ID NO 2) |
| EAKAEAKAEAKAEAKA,; | (SEQ ID NO 3) |
| KAEAKAEAKAEAKAEA,; | (SEQ ID NO 4) |
| AEAKAEAKAEAKAEAK,; | (SEQ ID NO 5) |
| ADADARARADADARAR,; | (SEQ ID NO 6) |
| ARADARADARADARAD,; | (SEQ ID NO 7) |
| DARADARADARADARA,; | (SEQ ID NO 8) |
| RADARADARADARADA,; | (SEQ ID NO 9) |
| ADARADARADARADAR,; | (SEQ ID NO 10) |
| ARADAKAE ARADAKAE,; | (SEQ ID NO 11) |
| AKAEARADAKAKARAD,; | (SEQ ID NO 12) |
| ARAKADAEARAKADAE,; | (SEQ ID NO 13) |
| AKARAEADAKARAEAD,; | (SEQ ID NO 14) |
| AQAQAQAQAQAQAQAQ,; | (SEQ ID NO 15) |
| VQVQVQVQVQVQVQVQ,; | (SEQ ID NO 16) |
| YQYQYQYQYQYQYQYQ,; | (SEQ ID NO 17) |
| HQHQHQHQHQHQHQHQ,; | (SEQ ID NO 18) |
| ANANANANANANANAN,; | (SEQ ID NO 19) |
| VNVNVNVNVNVNVNVN,; | (SEQ ID NO 20) |
| YNYNYNYNYNYNYNYN,; | (SEQ ID NO 21) |
| HNHNHNHNHNHNHNHN,; | (SEQ ID NO 22) |
| ANAQANAQANAQANAQ,; | (SEQ ID NO 23) |
| AQANAQANAQANAQAN,; | (SEQ ID NO 24) |
| VNVQVNVQVNVQVNVQ,; | (SEQ ID NO 25) |
| VQVNVQVNVQVNVQVN,; | (SEQ ID NO 26) |
| YNYQYNYQYNYQYNYQ,; | (SEQ ID NO 27) |
| YQYNYQYNYQYNYQYN,; | (SEQ ID NO 28) |
| HNHQHNHQHNHQHNHQ,; | (SEQ ID NO 29) |
| HQHNHQHNHQHNHQHN,; | (SEQ ID NO 30) |
| AKAQADAKAQADAKAQAD,; | (SEQ ID NO 31) |
| VKVQVDVKVQVDVKVQVD,; | (SEQ ID NO 32) |
| YKYQYDYKYQYDYKYQYD,; | (SEQ ID NO 33) |
| HKHQHDHKHQHDHKHQHD,; | (SEQ ID NO 34) |
| RARADADARARADADA,; | (SEQ ID NO 35) |
| RADARGDARADARGDA,; | (SEQ ID NO 36) |
| RAEARAEARAEARAEA,; | (SEQ ID NO 37) |
| KADAKADAKADAKADA,; | (SEQ ID NO 38) |
| AEAEAHAHAEAEAHAHA,; | (SEQ ID NO 39) |
| FEFEFKFKFEFEFKFK,; | (SEQ ID NO 40) |
| LELELKLKLELELKLK,; | (SEQ ID NO 41) |
| AEAEAKAKAEAEAKAK,; | (SEQ ID NO 42) |
| AEAEAEAEAKAK,; | (SEQ ID NO 43) |
| KAKAKAKAEAEAEAEA,; | (SEQ ID NO 44) |
| AEAEAEAEAKAKAKAK,; | (SEQ ID NO 45) |
| RARARARADADADADA,; | (SEQ ID NO 46) |
| ADADADADARARARAR,; | (SEQ ID NO 47) |
| DADADADARARARARA,; | (SEQ ID NO 48) |
| HEHEHKHKHEHEHKHK,; | (SEQ ID NO 49) |
| VEVEVEVEVEVEVEVEVEVE,; | (SEQ ID NO 50) |
| and | |
| RFRFRFRFRFRFRFRFRFRF,. | (SEQ ID NO 51) |

19. The method for improving myocardial infarction by intramyocardial or transendocardial injection of peptide nanofibers according to claim 18, wherein the self-assembling peptide nanofibers are RARADADARARADADA (SEQ ID NO 35), ADADARARADADARAR (SEQ ID NO 6), ARADARADARADARAD (SEQ ID NO 7), DARADARADARADARA (SEQ ID NO 8), RADARADARADARADA (SEQ ID NO 9), ADARADARADARADAR (SEQ ID NO 10), RARARARADADADADA (SEQ ID NO 46), ADADADADARARARAR (SEQ ID NO 47) or DADADADARARARARA (SEQ ID NO 48).

20. A method for improving myocardial infarction by intramyocardial or transendocardial injection of peptide nanofibers with autologous stem cells, comprising:
providing a pharmaceutical composition comprising a biologically compatible peptide hydrogel formed by a plurality of self-assembling peptide nanofibers having alternating hydrophobic and hydrophilic amino acids which are complementary and structurally compatible to one another, and at least one type of autologous stem cells mixed with the self-assembling peptide nanofibers; and
administering the pharmaceutical composition to an entire infarcted area of myocardium tissue with myocardial infarction by intramyocardial or transendocardial injection.


SEQUENCE LISTING (51 sequences)

SEQ ID NO: 1 - protein, 16 aa, artificial
  synthetic_sequence_designed_for_self_assembly
AKAKAEAEAKAKAEAE

SEQ ID NO: 2 - protein, 16 aa, artificial
  synthetic_sequence_designed_for_self_assembly
AKAEAKAEAKAEAKAE

SEQ ID NO: 3 - protein, 16 aa, artificial
  synthetic_sequence_designed_for_self_assembly
EAKAEAKAEAKAEAKA

SEQ ID NO: 4 - protein, 16 aa, artificial
  synthetic_sequence_designed_for_self_assembly
KAEAKAEAKAEAKAEA

SEQ ID NO: 5 - protein, 16 aa, artificial
  synthetic_sequence_designed_for_self_assembly
AEAKAEAKAEAKAEAK

SEQ ID NO: 6 - protein, 16 aa, artificial
  synthetic_sequence_designed_for_self_assembly
ADADARARADADARAR

SEQ ID NO: 7 - protein, 16 aa, artificial
  synthetic_sequence_designed_for_self_assembly
ARADARADARADARAD

SEQ ID NO: 8 - protein, 16 aa, artificial
  synthetic_sequence_designed_for_self_assembly
DARADARADARADARA

SEQ ID NO: 9 - protein, 16 aa, artificial
  synthetic_sequence_designed_for_self_assembly
RADARADARADARADA

SEQ ID NO: 10 - protein, 16 aa, artificial
  synthetic_sequence_designed_for_self_assembly
ADARADARADARADAR

SEQ ID NO: 11 - protein, 16 aa, artificial
  synthetic_sequence_designed_for_self_assembly
ARADAKAEARADAKAE

SEQ ID NO: 12 - protein, 16 aa, artificial
  synthetic_sequence_designed_for_self_assembly
AKAEARADAKAKARAD

SEQ ID NO: 13 - protein, 16 aa, artificial
  synthetic_sequence_designed_for_self_assembly
ARAKADAEARAKADAE

SEQ ID NO: 14 - protein, 16 aa, artificial
  synthetic_sequence_designed_for_self_assembly
AKARAEADAKARAEAD

SEQ ID NO: 15 - protein, 16 aa, artificial
  synthetic_sequence_designed_for_self_assembly
AQAQAQAQAQAQAQAQ

SEQ ID NO: 16 - protein, 16 aa, artificial
  synthetic_sequence_designed_for_self_assembly
VQVQVQVQVQVQVQVQ

SEQ ID NO: 17 - protein, 16 aa, artificial
  synthetic_sequence_designed_for_self_assembly
YQYQYQYQYQYQYQYQ

SEQ ID NO: 18 - protein, 16 aa, artificial
  synthetic_sequence_designed_for_self_assembly
HQHQHQHQHQHQHQHQ

SEQ ID NO: 19 - protein, 16 aa, artificial
  synthetic_sequence_designed_for_self_assembly
ANANANANANANANAN

SEQ ID NO: 20 - protein, 16 aa, artificial
  synthetic_sequence_designed_for_self_assembly
VNVNVNVNVNVNVNVN

SEQ ID NO: 21 - protein, 16 aa, artificial
  synthetic_sequence_designed_for_self_assembly
YNYNYNYNYNYNYNYN

SEQ ID NO: 22 - protein, 16 aa, artificial
  synthetic_sequence_designed_for_self_assembly
HNHNHNHNHNHNHNHN

SEQ ID NO: 23 - protein, 16 aa, artificial
  synthetic_sequence_designed_for_self_assembly
ANAQANAQANAQANAQ

SEQ ID NO: 24 - protein, 16 aa, artificial
  synthetic_sequence_designed_for_self_assembly
AQANAQANAQANAQAN

SEQ ID NO: 25 - protein, 16 aa, artificial
  synthetic_sequence_designed_for_self_assembly
VNVQVNVQVNVQVNVQ

SEQ ID NO: 26 - protein, 16 aa, artificial
  synthetic_sequence_designed_for_self_assembly
VQVNVQVNVQVNVQVN

SEQ ID NO: 27 - protein, 16 aa, artificial
  synthetic_sequence_designed_for_self_assembly
YNYQYNYQYNYQYNYQ

SEQ ID NO: 28 - protein, 16 aa, artificial
  synthetic_sequence_designed_for_self_assembly
YQYNYQYNYQYNYQYN

SEQ ID NO: 29 - protein, 16 aa, artificial
  synthetic_sequence_designed_for_self_assembly
HNHQHNHQHNHQHNHQ

SEQ ID NO: 30 - protein, 16 aa, artificial
  synthetic_sequence_designed_for_self_assembly
HQHNHQHNHQHNHQHN

SEQ ID NO: 31 - protein, 18 aa, artificial
  synthetic_sequence_designed_for_self_assembly
AKAQADAKAQADAKAQAD

SEQ ID NO: 32 - protein, 18 aa, artificial
  synthetic_sequence_designed_for_self_assembly
VKVQVDVKVQVDVKVQVD

SEQ ID NO: 33 - protein, 18 aa, artificial
  synthetic_sequence_designed_for_self_assembly
YKYQYDYKYQYDYKYQYD

SEQ ID NO: 34 - protein, 18 aa, artificial
  synthetic_sequence_designed_for_self_assembly
HKHQHDHKHQHDHKHQHD

SEQ ID NO: 35 - protein, 16 aa, artificial
  synthetic_sequence_designed_for_self_assembly
RARADADARARADADA

SEQ ID NO: 36 - protein, 16 aa, artificial
  synthetic_sequence_designed_for_self_assembly
RADARGDARADARGDA

SEQ ID NO: 37 - protein, 16 aa, artificial
  synthetic_sequence_designed_for_self_assembly
RAEARAEARAEARAEA

SEQ ID NO: 38 - protein, 16 aa, artificial
  synthetic_sequence_designed_for_self_assembly
KADAKADAKADAKADA

SEQ ID NO: 39 - protein, 17 aa, artificial
  synthetic_sequence_designed_for_self_assembly
AEAEAHAHAEAEAHAHA

SEQ ID NO: 40 - protein, 16 aa, artificial
  synthetic_sequence_designed_for_self_assembly
FEFEFKFKFEFEFKFK

SEQ ID NO: 41 - protein, 16 aa, artificial
  synthetic_sequence_designed_for_self_assembly
LELELKLKLELELKLK

SEQ ID NO: 42 - protein, 16 aa, artificial
  synthetic_sequence_designed_for_self_assembly
AEAEAKAKAEAEAKAK

SEQ ID NO: 43 - protein, 12 aa, artificial
  synthetic_sequence_designed_for_self_assembly
AEAEAEAEAKAK

SEQ ID NO: 44 - protein, 16 aa, artificial
  synthetic_sequence_designed_for_self_assembly
KAKAKAKAEAEAEAEA

SEQ ID NO: 45 - protein, 16 aa, artificial
  synthetic_sequence_designed_for_self_assembly
AEAEAEAEAKAKAKAK

SEQ ID NO: 46 - protein, 16 aa, artificial
  synthetic_sequence_designed_for_self_assembly
RARARARADADADADA

SEQ ID NO: 47 - protein, 16 aa, artificial
  synthetic_sequence_designed_for_self_assembly
ADADADADARARARAR

SEQ ID NO: 48 - protein, 16 aa, artificial
  synthetic_sequence_designed_for_self_assembly
DADADADARARARARA

SEQ ID NO: 49 - protein, 16 aa, artificial
  synthetic_sequence_designed_for_self_assembly
HEHEHKHKHEHEHKHK

SEQ ID NO: 50 - protein, 20 aa, artificial
  synthetic_sequence_designed_for_self_assembly
VEVEVEVEVEVEVEVEVEVE

SEQ ID NO: 51 - protein, 20 aa, artificial
  synthetic_sequence_designed_for_self_assembly
RFRFRFRFRFRFRFRFRFRF